(19) United States
(12) Patent Application Publication
(10) Pub. No.: US 2025/0040832 A1
(43) Pub. Date: Feb. 6, 2025

(54) ACCESSIBILITY DIAGNOSIS FOR COSMETIC APPLICATOR CONFIGURED FOR USERS WITH LIMITED MOBILITY

(71) Applicant: L'OREAL, Paris (FR)

(72) Inventors: Casey BARBARINO, San Anselmo, CA (US); Gregoire CHARRAUD, Jersey City, NJ (US); Hyosik Dennis MIN, Palisades Park, NJ (US)

(73) Assignee: L'OREAL, Paris (FR)

(21) Appl. No.: 18/228,347

(22) Filed: Jul. 31, 2023

(51) Int. Cl. A61B 5/11; A45D 44/00

(52) U.S. Cl. CPC A61B 5/1101; A45D 44/00; A45D 2200/10

(57) ABSTRACT

An accessibility diagnosis system is provided for a motion stabilization device for stabilization of a cosmetic applicator. The system includes a mobile user device that includes a touch sensitive display and a motion sensor, the mobile user device including processing circuitry configured to: display a request to a user for an input of a specific drawing motion or a specific movement of the mobile user device, and collect information indicating a movement pattern of the user's hands based on the results of the user's input in response to the request; and a server device that is connected to the mobile user device via a network, the server device including processing circuitry configured to receive the collected information from the mobile user device, determine an amount of motion stabilization required to be set for the motion stabilization device based on the collected information, and output the determined amount of motion stabilization.

### FIELD

The present disclosure describes a system and features related to a device for modifying, mitigating, altering, reducing, compensation for, or the like, the movement of a cosmetic applicator caused by unintentional movements, tremors, limited mobility, or the like of a user.

### BACKGROUND

Unintentional movements of the human body, or human tremors, can occur in individuals suffering from motion disorders or even healthy individuals. Due to these unintentional movements, a person may have difficulty in performing a task that requires care and precision, such as applying a cosmetic composition to a part of the body, such as the face, hands, or feet.

Therefore, there is a need for a solution that allows application of a cosmetic composition that is compatible with the diverse and disposable nature of cosmetic applicators.

### SUMMARY

In an embodiment, an accessibility diagnosis system is provided for a motion stabilization device for stabilization of a cosmetic applicator, comprising: a mobile user device that includes a touch sensitive display and a motion sensor, the mobile user device including processing circuitry configured to: display a request to a user for an input of a specific drawing motion or a specific movement of the mobile user device, and collect information indicating a movement pattern of the user's hands based on the results of the user's input in response to the request; and a server device that is connected to the mobile user device via a network, the server device including processing circuitry configured to receive the collected information from the mobile user device, determine an amount of motion stabilization required to be set for the motion stabilization device based on the collected information, and output the determined amount of motion stabilization.

In an embodiment, the input of a specific drawing motion includes at least one of drawing a straight line and drawing a circular pattern.

In an embodiment, the input of a specific movement of the mobile user device includes at least one of moving the mobile user device close to the user's face and rotating the mobile user device in a predetermined direction.

In an embodiment, the mobile user device is configured to provide a questionnaire to the user and the amount of motion stabilization required to be set for the motion stabilization device is further based on the user's responses to the questionnaire.

In an embodiment, the processing circuitry of the server device is configured to combine the collected information from the mobile user device and determine if motion stabilization is active or static based on comparing a value associated with the collected information to a threshold.

In an embodiment, the processing circuitry of the server device is configured to determine if motion stabilization is active or static for at least one particular cosmetic application based on comparing a value associated with the collected information to a threshold.

In an embodiment, the processing circuitry server device determines the amount of motion stabilization by inputting the collected information into a neural network that is trained to output a motion stabilization profile based on pre-existing combinations collected information the motion stabilization profiles.

### BRIEF DESCRIPTION OF THE DRAWINGS

A more complete appreciation of the embodiments and many of the attendant advantages thereof will be readily obtained as the same becomes better understood by reference to the following detailed description when considered in connection with the accompanying drawings, wherein:

FIG. 1 shows a motion stabilizing device.

FIG. 2 shows how the motion stabilizing device couples with an adaptor and a make-up applicator.

FIG. 3A shows a diagram of the internal components of motion stabilizing device.

FIG. 3B shows a diagram of an alternative embodiment of the motion stabilizing device in which the receiver portion includes an electromagnetic positioner instead of the motive elements shown in FIG. 3A.

FIG. 4 shows an overview of a universal adapter handle connection system.

FIG. 5 shows a system according to an embodiment that includes a mobile user device, a motion stabilizer, and a server device.

FIGS. 6A and 6B show two challenges involving the user attempting to draw objects on the smartphone itself.

FIGS. 6C and 6D show examples of how results are detected based on the challenges presented in FIGS. 6A and 6C.

FIGS. 7A-7D show four challenges related to moving the smartphone itself in space.

FIGS. 7E and 7F show how the results are received for any of the challenges shown on FIGS. 7A-7D.

FIG. 8 shows an additional information of collecting information about the user with a questionnaire.

FIG. 9 shows that at the server device the results of the various challenges are input to a diagnostic engine.

FIG. 10 illustrates that one method of applying data collection to the motion stabilizer is to make binary decision on whether to use the motion stabilizer or not during certain types of cosmetic applications.

FIG. 11 shows a process that is specifically based on the results of the challenge shown in FIG. 6A.

FIG. 12 shows a process that is specifically based on the results of the challenge shown in FIG. 61, where a user is asked to draw a circle.

FIG. 13 is a diagram shown how machine learning can help determine motion stabilization settings.

FIG. 14 shows the usage of the deep learning model after training has reached an adequate level.

FIG. 15 shows an example of a display to a user based on the results of the diagnosis in the form of a “fit graph.’

FIG. 16 is a more detailed block diagram illustrating a mobile user device according to certain embodiments of the present disclosure.

FIG. 17 shows a hardware description of the server device.

### DETAILED DESCRIPTION

The present disclosure describes a cosmetic applicator system that minimizes modifies, mitigates, alters, reduces, compensates for, or the like unintentional movements by stabilizing, orienting, operating, controlling, etc. an applicator for a user and is also designed to be flexible to accommodate different types of commercially available cosmetic applications. The present disclosure further describes a system and features to enhance the functionality of such a cosmetic applicator system.

The basic features and operation of a motion stabilizing device for a cosmetic applicator is described in U.S. Pat. No. 11,458,062, which is incorporated herein by reference.

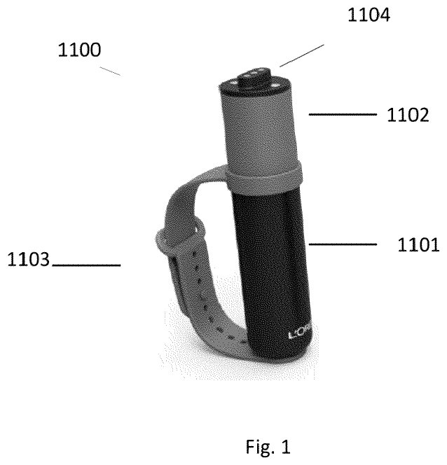

FIG. 1 shows a conventional motion stabilizing device 1100, which serves as a base unit for receiving a cosmetic applicator according to an embodiment. The device 1100 includes a handle portion 1101, a receiver portion 1102 and a strap 1103. The receiver portion 1102 includes an interface 1104, shown as a male connector that couples with a cosmetic applicator, which will be discussed in detail below. The receiver portion could be utilized for communication between the base unit and the applicator. The connection to an adaptor and/or an applicator could be accomplished with a mechanical coupling, such as screw-in or snap-fit, or it could be accomplished with magnets.

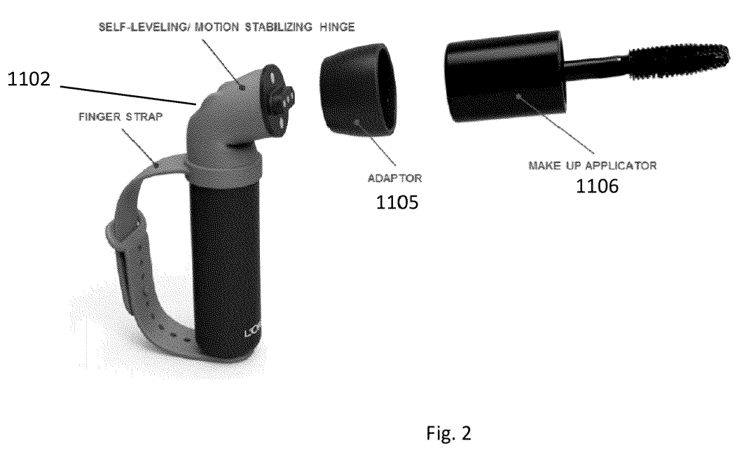

FIG. 2 shows how the device 1100 couples with an adaptor 1105 and a make-up applicator 1106. It can be seen that the adaptor fits over the exposed end of the receiver portion 1102. The adaptor includes electrical mating connectors (a female connector—not shown) in a recessed portion to make contact with the electric interface of the receiver portion 1101.

As shown in FIG. 2, the receiver portion 1102 is configured to contort, articulate, reposition, etc., between an upright posture (as shown in FIG. 1) and an angled posture (as shown in FIG. 2). This is accomplished with a hinge mechanism contained inside the receiver portion 1102. FIG. 2 shows that the hinge mechanism is a self-leveling/motion stabilizing hinge.

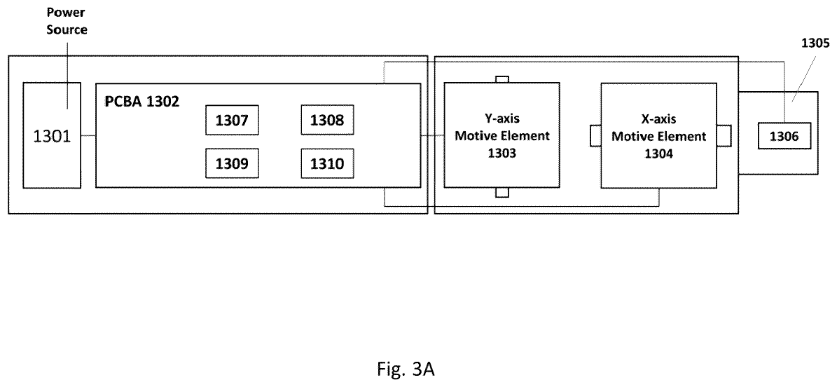

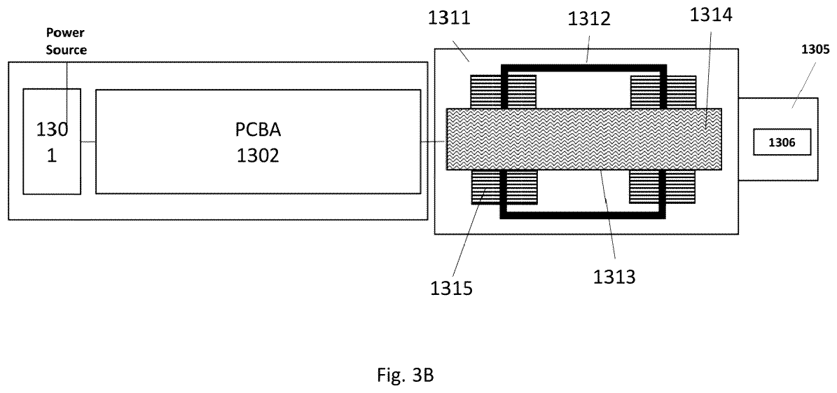

FIG. 3A shows a diagram of the internal components of device 1100 according to one embodiment. In the handle portion, the device includes a power source 1301, which may be a battery or the like. The device includes a printed circuit board assembly (PCBA) 1302, which may include positional sensor circuitry 1307, reader circuitry 1308, control circuitry 1309, and communication interface 1310, as understood in the art.

For instance, as the sensor circuitry 1307, the PCBA may include at least one inertial sensor and at least one distributed motion sensor to detect unintentional muscle movements and measure signals related to these unintentional muscle movements that are created when a user adversely affects motion of the applicator. These sensors also detect the motion of the stabilized output relative to device. The control circuitry sends voltage commands in response to the signals to the motion generating elements (described below) to cancel the user's tremors or unintentional muscle movements. This cancellation maintains and stabilizes a position of the applicator, keeping it stable.

One of ordinary skill in the art readily recognizes that a system and method in accordance with the present invention may utilize various implementations of the control circuitry and the sensor circuitry and that would be within the spirit and scope of the present invention. In one embodiment, the control circuitry 1309 comprises an electrical system capable of producing an electrical response from sensor inputs such as a programmable microcontroller or a field-programmable gate array (FPGA). In one embodiment, the control circuitry comprises an 8-bit ATMEGA8A programmable microcontroller manufactured by Atmel due to its overall low-cost, low-power consumption and ability to be utilized in high-volume applications.

In one embodiment, the at least one inertial sensor in the sensor circuitry is a sensor including but not limited to an accelerometer, gyroscope, or combination of the two. In one embodiment, the at least one distributed motion sensor in the sensor circuitry is a contactless position sensor including but not limited to a hall-effect magnetic sensor.

The system created by the combination of the sensor circuitry, the control circuitry, and the motion generating elements may be a closed-loop control system that senses motion and acceleration at various points in the system and feeds detailed information into a control algorithm that moves the motion-generating elements appropriately to cancel the net effect of a user's unintentional muscle movements and thus stabilize the position of the applicator. The operation and details of the elements of the control system and control algorithm are understood in the art, as described in U.S. PG Publication 2014/0052275A1, incorporated herein by reference.

The communication interface 1310 may include a network controller such as BCM43342 Wi-Fi, Frequency Modulation, and Bluetooth combo chip from Broadcom, for interfacing with a network.

In the receiver portion of the device, there may be two motive elements to allow 3-dimensional movement of the receiver as anti-shaking movement. The two motive elements include a y-axis motive element 1303 and an x-axis motive element 1304, each being connected to and controlled by the PCBA 1302. Each of the motive elements may be servo motors as understood in the art. The device further includes end effector coupling 1305, which is configured to couple with the adaptor 1105. The end effector coupling 1305 may include a radiofrequency identification (RFID)) reader 1306, configured to read an RFID tag, which may be included with the applicator, as will be discussed below.

FIG. 3B shows a diagram of an alternative embodiment of the device 1100 in which the receiver portion includes an electromagnetic positioner 1311 instead of the motive elements shown in FIG. 3A. The electromagnetic positioner 1311 may include U-shaped magnetic cores 1312 arrayed around a non-magnetic tube 1313, which is filled with a magnetic fluid 1314. Each of the magnetic cores has arm portions that are surrounded by windings 1315. The magnetic cores may be controlled by the control circuitry in the PCBA 1302 to act as a controllable active magnetic field-generating structure which is used to generate a variable magnetic field that acts upon the magnetic fluid, causing it to be displaced, thereby enabling the armature to be moved to a desired coordinate position and/or orientation. The details of implementing the electromagnetic positioner 1311 may be found in U.S. Pat. No. 6,553,161, which is incorporated herein by reference.

In the above-described conventional motion stabilizing device, there is a problem that the interface 1104 that receives the adaptor 1105 requires a specific point of attachment to align properly with the interface.

Therefore, the below embodiments provide a universal adapter connection between the handle of the motion stabilizing device in order to improve user experience and reduce the struggle and time taken to set up the system for use.

In one embodiment, the present disclosure is directed towards a cosmetic applicator. The cosmetic applicator can be used for a variety of cosmetics and cosmetic applications, including, but not limited to, mascara, eyeliner, eyebrow products, lip products (lipstick, lip gloss, lip liner, etc.), skin products, and/or hair products. In one embodiment, the cosmetic applicator can include an adapter, wherein the adapter can connect the cosmetic applicator to a motion stabilizer. The motion stabilizer can be, for example, a handle that can counteract unintentional motions such as tremors or spasms. These motions can interfere with the application of cosmetics and can also make it difficult to generally interact with cosmetic applicators or tools. For example, the many cosmetic products require a twisting motion or force to be applied to open or extrude the product. It can be difficult for users to achieve the range of motion or the precision necessary to apply these forces to the cosmetic. In one embodiment, the cosmetic applicator can hold a cosmetic and can enable the proper force to be applied to the cosmetic to open, close, mix, stir, blend, extrude, or achieve other similar functions necessary for application.

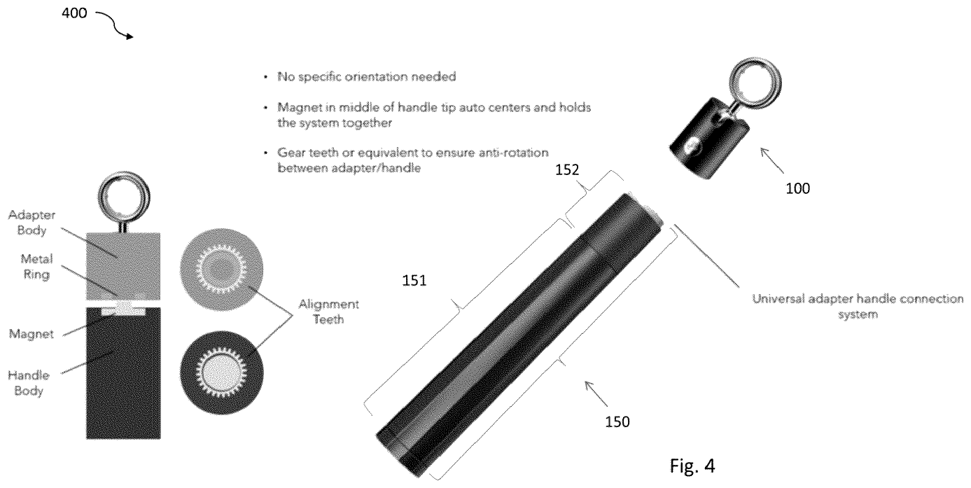

FIG. 4 shows an overview of a universal adapter handle connection system 400. The system includes a motion stabilizer device 150 that includes a handle portion 151 and a hinge portion 152 (receiver portion) that is functionally similar to the device 1100 shown in FIG. 1. It further includes a universal adapter 100 that attaches to the device 150 and also holds different types of cosmetic applicators. The basic features and operation of universal adapter handle connection system 400 are described in co-pending U.S. application Ser. Nos. 18/091,882; 18/091,920; 18/091,843; 18/091,925; 18/148,957; 18/148,880; and 18/148,930, which are incorporated herein by reference.

For the above-described device, due to different limitations caused by different disabilities, it can be challenging to understand what level of assistance is needed to provide a helpful/accurate assisted makeup application experience (i.e. how many degrees of freedom & how much automation from device). The below embodiments provide a simple self-guided diagnostic tool for at home use. Users use an online or app-based tool to run through simple exercises that will help diagnose how much assistance they need from the device, and can make a recommendation on which options of device best suit their needs (if any).

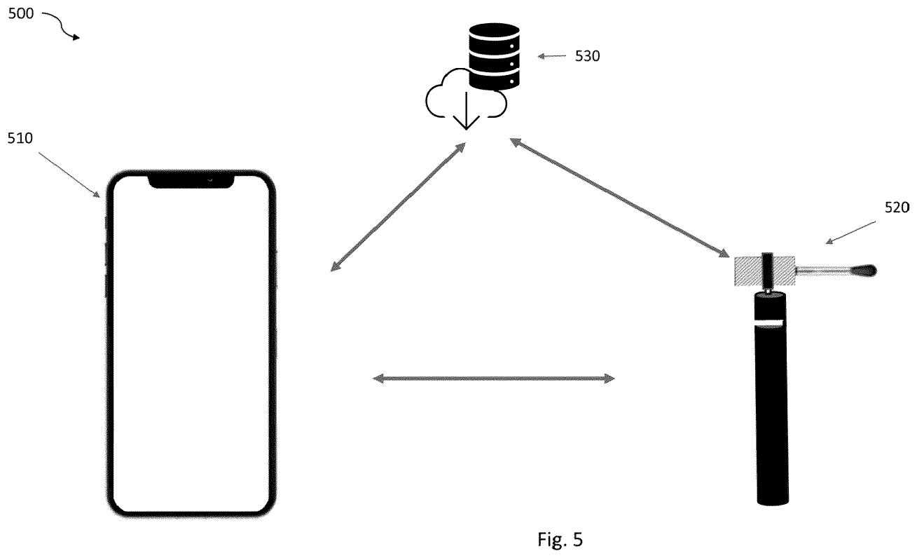

FIG. 5 shows a system 500 according to an embodiment includes a mobile user deice 510 and the motion stabilizer 520 described above. The system further includes a cloud server device 530 that connects to both the smartphone and the motion stabilizer. The smartphone will provide a user with multiple challenges and possibly a questionnaire to diagnose how much assistance they will need in terms of motion stabilization when using the device.

The system measures: shaking, duration to accomplish challenge, pattern accuracy, detects phone fall, asks about pain/discomfort, and records sound. Specifically, the smartphone is utilized to ask users to perform motions such as: draw a line (tremor check), draw a circle (prediction check), bring smartphone to face (elbow mobility check), rotation from portrait to landscape (wrist mobility check), detect fall (finger grasping check). The smartphone records position in real time utilizing accelerometer/gyroscope/camera.

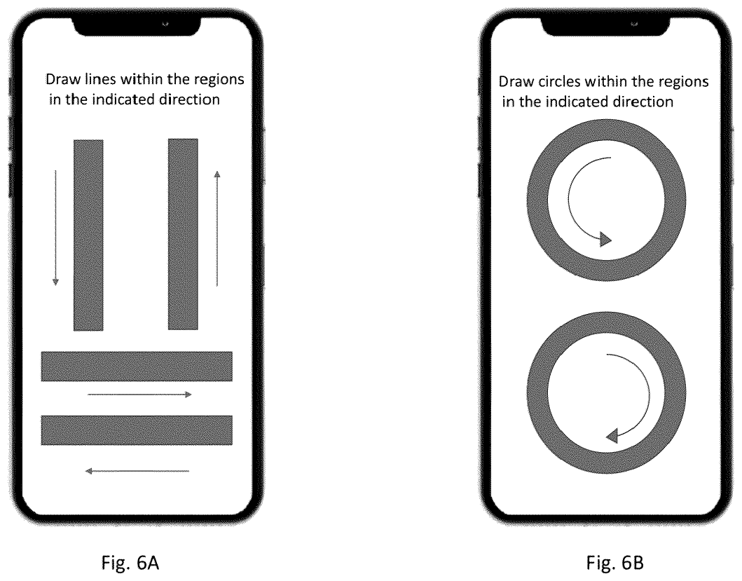

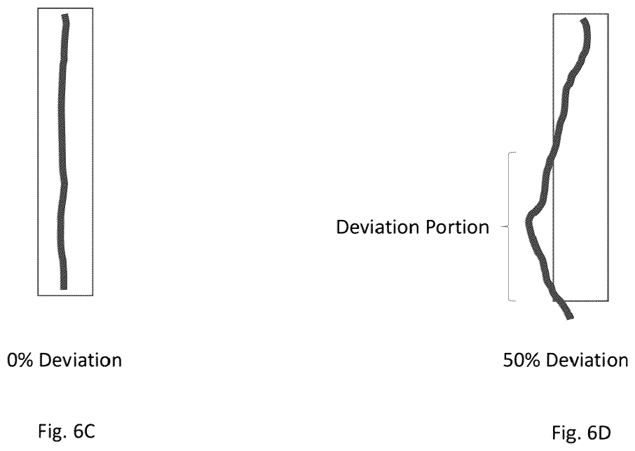

FIGS. 6A and 6B show two challenges involving the user attempting to draw objects on the smartphone itself. FIG. 6A requests the user to draw straight lines, and FIG. 6B requests the user to draw circular patterns. A target shape may be displayed which the user will attempt to trace within a shaped region.

FIGS. 6C and 6D show examples of how results are detected based on the challenges presented in FIGS. 6A and 6C. As shown in FIGS. 6A and 6B, the target shape has a certain thickness that the user is essentially drawing within. The centroid of the user's contact on the touchscreen is detected, as is understood in the art. FIG. 6C shows an example when the user drawings a fairly straight line that stays with the displayed target region. When the user keeps the line within the region, it may be considered to have 0% deviation. On the contrary, FIG. 6D shows an example when the user is unable to keep the line straight and within the displayed region. In this situation, half of the user's contact is outside the region and thus this represents 50% deviation. The same method of detecting deviation applies to the circle example shown in FIG. 6B.

While the above method describes one way to detect if a user has successfully drawn a line or a circle, other methods may be used as is understood in the art. For instance, a method of recognizing graphical objects, such as lines and circles, is described in U.S. Pat. No. 10,372,321, which is incorporated herein by reference. Additionally, image recognition techniques may be used to determine if the user has successfully drawn a recognizable line or circle.

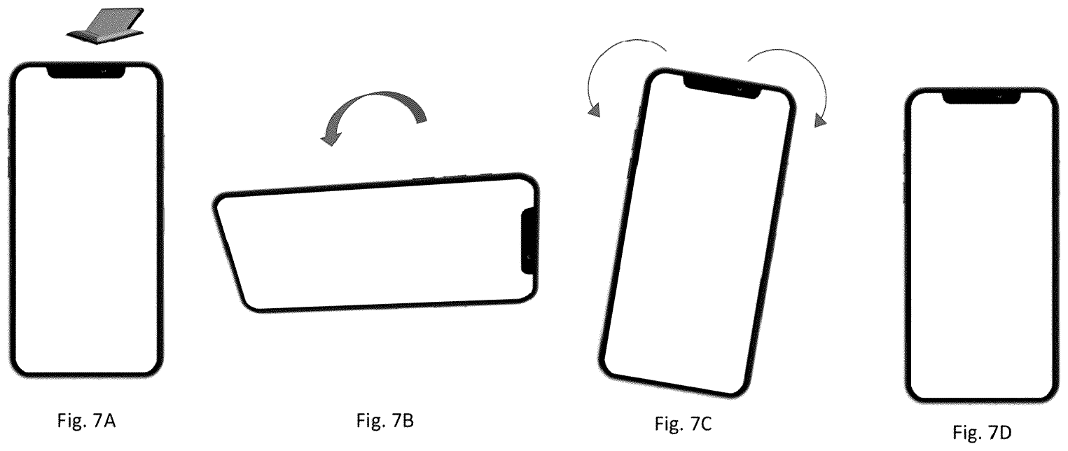

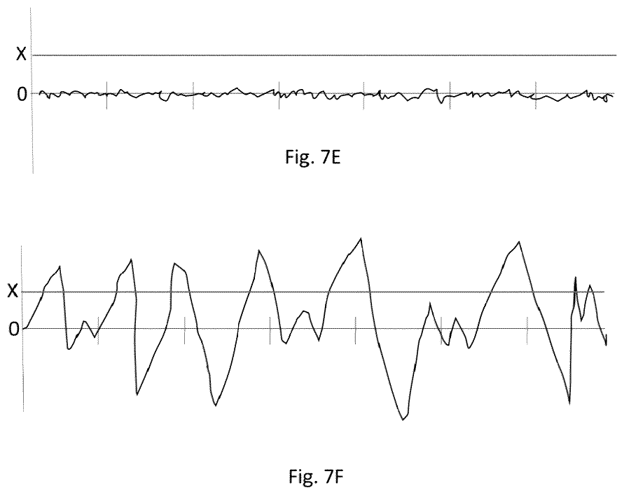

FIGS. 7A-7D show four challenges related to moving the smartphone itself in space. FIG. 7A shows a challenge in which the user is asked to bring the phone to close to a target area, such as the lips or eyes. This challenge aims to show a user's ability to control a device while bringing it towards their face.

FIG. 7B shows a challenge where the user is asked to attempt to rotate the smartphone forward or backwards in landscape mode. FIG. 7C shows a challenge where the user is asked to rotate the smartphone clockwise or counter-clockwise. These challenges aims to show a user's ability to make wrist movements while operating the device during application of the make-up.

FIG. 7D shows a challenge where the user is asked to hold the smartphone still. This challenge aims to determine the level of unintentional movements of the user's hands when just holding the device in space.

FIGS. 7E and 7F show how the results are received for any of the challenges shown on FIGS. 7A-7D. FIG. 7E shows the amplitude of vibration for a user that does not show signs of unintentional movements of the hands when performing any of the challenges. However, FIG. 7F shows the amplitude of vibration for a user that does show signs of unintentional movements of the hands when performing any of the challenges. As can be seen, in FIG. 7F, the amplitude of vibration is above a threshold amount X.

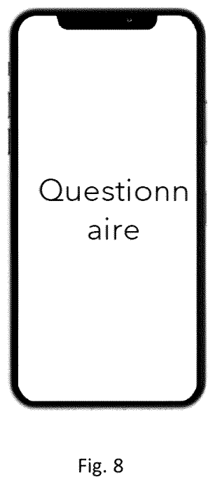

FIG. 8 shows an additional information of collecting information about the user with a questionnaire. Such questions may request age, gender, and health conditions. Additionally, if will be asked if the user has a particular health condition that affects the movement of their hands.

The collection of data from the various tests and questionnaire may come in different forms. For instance, a percentage accuracy of staying on the lines when tracing lines or circles may be collected. Additionally, the nature of the tracing (such as a “shaky’ tracing pattern) will be detected as well.

Similarly, the shakiness of the smartphone while the user is attempting to bring the smartphone to a target area on the face, or when rotational exercises are being performed, will be detected.

The data that is collected during the challenges is transmitted to the server device 530. The server device will analyze the data to determine an amount and type of support needed to be provided by the motion stabilizer device.

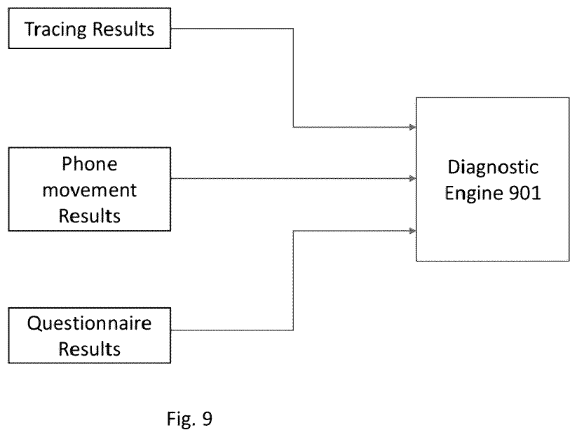

FIG. 9 shows that at the server device 530 the results of the various challenges are input to a diagnostic engine 901, which is implemented by processing circuitry at the server device 530.

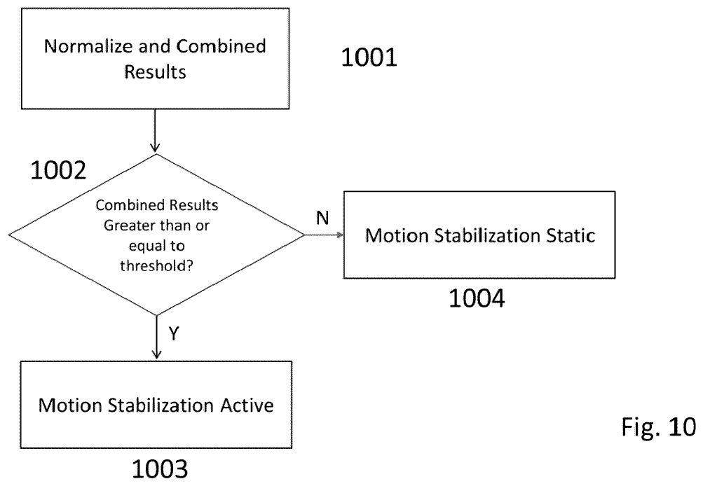

FIG. 10 illustrates that one method of applying data collection to the motion stabilizer is to make binary decision on whether to use the motion stabilizer or not during certain types of cosmetic applications. In Step 1001, the results that are input to the diagnostic engine are normalized and combined. For instance, all results can be transformed into an integer value. When the user is attempting to trace the line or circle, each time the user deviated from the path will increment a counter to give an integer value. For the challenges when the smartphone is being moved closer to the face or rotated in landscape mode, then each time the user shakes the phone in the process will increment a counter. Also, certain questions to the questionnaire will cause a counter to be incremented. For example, if the user answers “yes” to having tremors, then that will increment a counter.

In Step 1002, the combined integer value from all of the counters is compared to a threshold number. This number can be predetermined based on combined data from multiple users. In Step 1003, if the number is greater than or equal to the threshold number, then it is determined that motion stabilization should be activated. In Step 1004, if the number is not greater than or equal to the threshold number, then motion stabilization should not be activated (i.e., the device should be static).

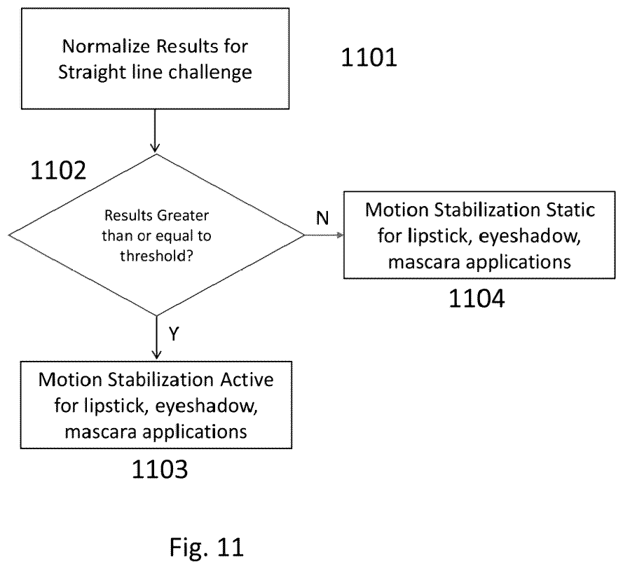

While the above process of FIG. 10 represents a simplified method of determining a binary setting for the motion stabilization function on the device, a customized approach may also be used. For instance, certain results are indicative that the user may need motion stabilization for one application but not another. FIG. 11 shows a process that is specifically based on the results of the challenge shown in FIG. 6A. In step 1101, the results for the straight line challenge are normalized. In step 1102, the results are compared to a threshold value. Then in step 1103 or 1104, motion stabilization will be active or deactivated for lipstick, eyeshadow, or mascara applications based on the results.

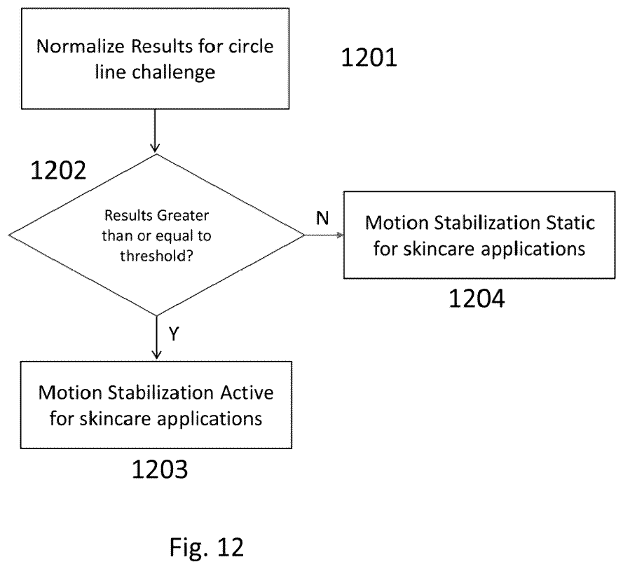

Alternatively, FIG. 12 shows a process that is specifically based on the results of the challenge shown in FIG. 6B, where a user is asked to draw a circle. In step 1201, the results for the circle line challenge are normalized. In step 1202, the results are compared to a threshold value. Then in step 1203 or 1204, motion stabilization will be active or deactivated for skincare applications.

The above processes show considerations by the diagnostic engine in setting the motion stabilizer. However, if the results show severe shakiness from either of the challenges in FIG. 7A or 7B, then the diagnostic engine may override the results of the processes in FIG. 11 or 12, and still activate motion stabilization.

Alternatively, certain parameters within the motion stabilizer may be set based on the results obtained from the diagnostic engine. For instance, the results could be used to turn on/off different variables, or weight variables, related to rotation or flexion. Since a specific user profile may be complex to customize, machine learning may also be used to determine the optimum settings for a user.

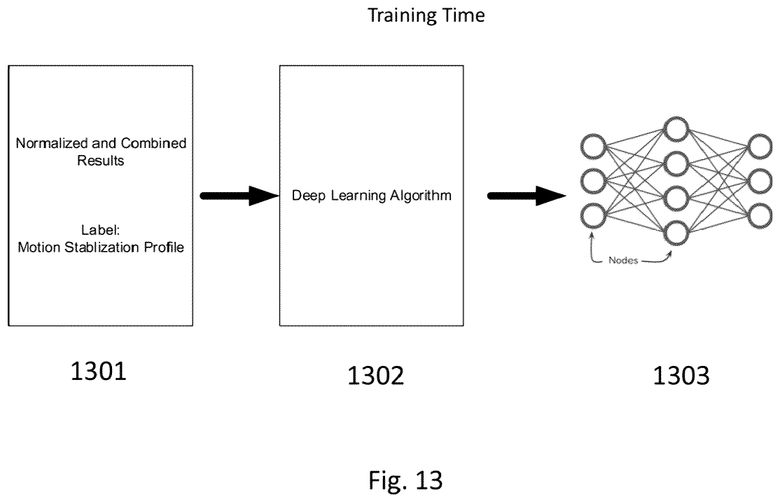

FIG. 13 is a diagram shown how machine learning can help determine motion stabilization settings. During training, previous users data is collected that has a combination of results of the diagnostic challenges described above and the optimal motion stabilization settings profile that was used for a respective user. These inputs are provided at stage 1301, where normalized and combined results similar to those described above are input along with the motion stabilization profile that essentially acts as a label.

The inputs are provided to a deep learning algorithm in step 1302. The deep learning algorithm used may be based on available software as known in the art, such as Tensorflow, Keras, Mxnet, Caffe, or Pytorch. The result of the labeled training will be a neural network at step 1303. The neural network created includes nodes of each layer are clustered, the clusters overlap, and each cluster feeds data to multiple nodes of the next layer.

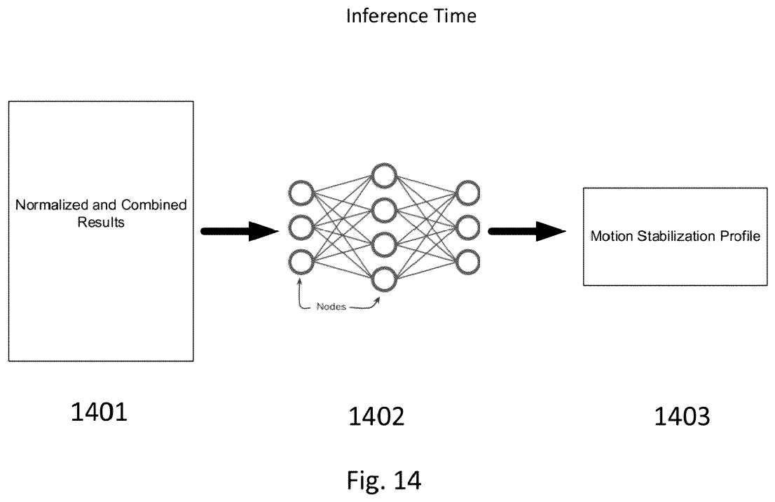

FIG. 14 shows the usage of the deep learning model after training has reached an adequate level. This is referred to as “inference time” since recommendation will be inferred from input data without a label. It can be seen that the input stage does not include a label of a motion stabilization profile. The inputs are fed to the trained neural network at step 1402, which will provide an output at step 1403 of the motion stabilization profile that best fits the inputted normalized and combined results of the user. With the neural network model described herein, complex diagnosis results can be utilized to determine a custom motion stabilization profile for a user without having to come with up predetermined manual settings.

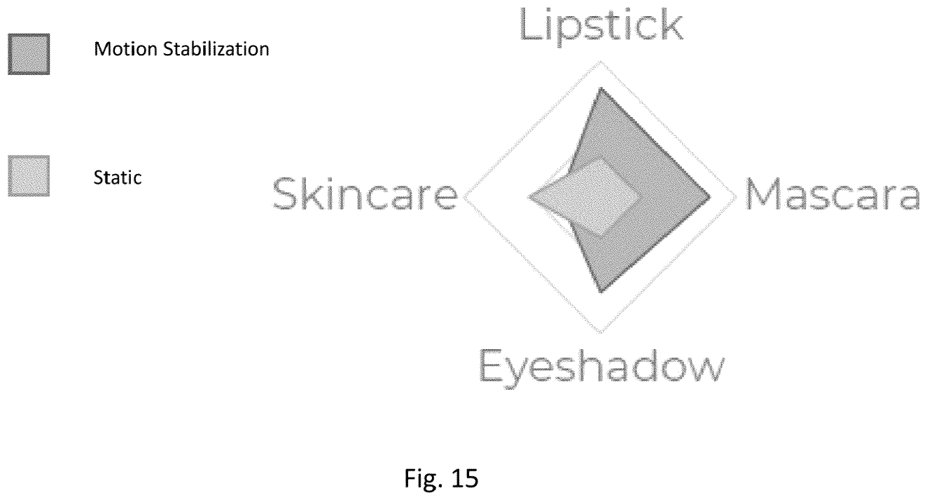

FIG. 15 shows an example of a display to a user based on the results of the diagnosis in the form of a “fit graph. As shown, different types of cosmetic applications may populate the points along a perimeter of the graph, and color coding can be used to indicate which of the cosmetic applications will have motion stabilization, and which will not.

The above-described embodiments allows for characterization of a user's level of mobility to check whether or not a motion stabilizer will be useful, which will allow the consumer to determine if the device will be helpful for them or not even prior to purchase.

Furthermore, while the above-described embodiments describe the functionality being performed by a server device 530, it will be appreciated that the functionality can be performed by the user device 510 or a personal computer (not shown) of the user.

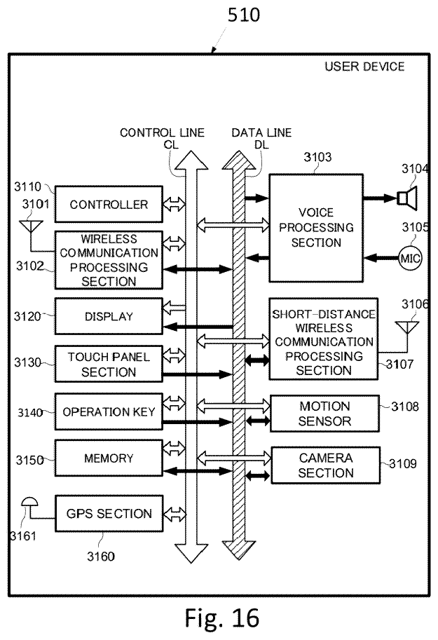

FIG. 16 is a more detailed block diagram illustrating a mobile user device 510 according to certain embodiments of the present disclosure. In certain embodiments, user device 510 is a smartphone. However, the skilled artisan will appreciate that the features described herein may be adapted to be implemented on other devices (e.g., a laptop, a tablet, a server, an e-reader, a camera, a navigation device, etc.). The exemplary user device 510 includes a controller 3110 and a wireless communication processor 3102 connected to an antenna 3101. A speaker 3104 and a microphone 3105 are connected to a voice processor 3103.

The controller 3110 may include one or more Central Processing Units (CPUs), and may control each element in the user device 510 to perform functions related to communication control, audio signal processing, control for the audio signal processing, still and moving image processing and control, and other kinds of signal processing. The controller 3110 may perform these functions by executing instructions stored in a memory 3150. Alternatively or in addition to the local storage of the memory 150, the functions may be executed using instructions stored on an external device accessed on a network or on a non-transitory computer readable medium. As described above in relation to FIG. 16, the controller 3110 may execute instructions allowing the controller 3110 to function as the display control unit 3211, operation management unit 3212 and game management unit 3213 depicted in FIG. 16.

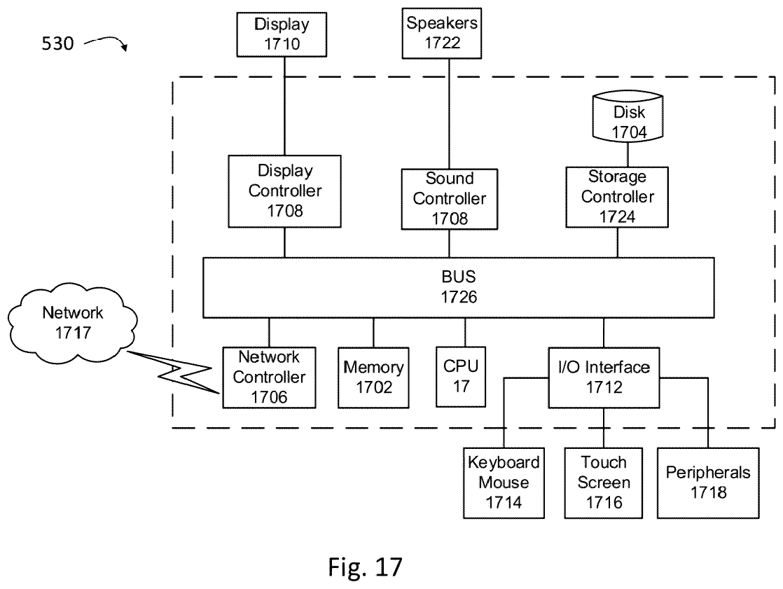

Next, a hardware description of the server device 530 according to exemplary embodiments is described with reference to FIG. 17. In FIG. 17, the server device 530 includes a CPU 1700 which performs the processes described above/below. The process data and instructions may be stored in memory 1702. These processes and instructions may also be stored on a storage medium disk 1704 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the [device] communicates, such as a server or computer.

Further, the claimed advancements may be provided as a utility application, background daemon, or component of an operating system, or combination thereof, executing in conjunction with CPU 1700 and an operating system such as Microsoft Windows 7, UNIX, Solaris, LINUX, Apple MAC-OS and other systems known to those skilled in the art.

The hardware elements in order to achieve the [device] may be realized by various circuitry elements, known to those skilled in the art. For example, CPU 1700 may be a Xenon or Core processor from Intel of America or an Opteron processor from AMD of America, or may be other processor types that would be recognized by one of ordinary skill in the art. Alternatively, the CPU 1700 may be implemented on an FPGA, ASIC, PLD or using discrete logic circuits, as one of ordinary skill in the art would recognize. Further. CPU 1700 may be implemented as multiple processors cooperatively working in parallel to perform the instructions of the inventive processes described above.

The [device] in FIG. 17 also includes a network controller 1706, such as an Intel Ethernet PRO network interface card from Intel Corporation of America, for interfacing with network 1717. As can be appreciated, the network 1717 can be a public network, such as the Internet, or a private network such as an LAN or WAN network, or any combination thereof and can also include PSTN or ISDN sub-networks. The network 1717 can also be wired, such as an Ethernet network, or can be wireless such as a cellular network including EDGE, 3G and 4G wireless cellular systems. The wireless network can also be WiFi, Bluetooth, or any other wireless form of communication that is known.

The [device] further includes a display controller 1708, such as a NVIDIA GeForce GTX or Quadro graphics adaptor from NVIDIA Corporation of America for interfacing with display 1710, such as a Hewlett Packard HPL2445w LC) monitor. A general purpose I/O interface 1712 interfaces with a keyboard and/or mouse 1714 as well as a touch screen panel 1716 on or separate from display 1710. General purpose I/O interface also connects to a variety of peripherals 1718 including printers and scanners, such as an OfficeJet or DeskJet from Hewlett Packard.

A sound controller 1720 is also provided in the [device), such as Sound Blaster X-Fi Titanium from Creative, to interface with speakers/microphone 1722 thereby providing sounds and/or music.

The general purpose storage controller 1724 connects the storage medium disk 1704 with communication bus 1726, which may be an ISA, EISA, VESA, PCI, or similar, for interconnecting all of the components of the [device]. A description of the general features and functionality of the display 1710, keyboard and/or mouse 1714, as well as the display controller 1708, storage controller 1724, network controller 1706, sound controller 1720, and general purpose I/O interface 1712 is omitted herein for brevity as these features are known.

Obviously, numerous modifications and variations of the present invention are possible in light of the above teachings. It is therefore to be understood that within the scope of the appended claims, the invention may be practiced otherwise than as specifically described herein.

1. An accessibility diagnosis system for a motion stabilization device for stabilization of a cosmetic applicator, comprising:
a mobile user device that includes a touch sensitive display and a motion sensor, the mobile user device including processing circuitry configured to:
display a request to a user for an input of a specific drawing motion or a specific movement of the mobile user device, and
collect information indicating a movement pattern of the user's hands based on the results of the user's input in response to the request; and
a server device that is connected to the mobile user device via a network, the server device including processing circuitry configured to
receive the collected information from the mobile user device,
determine an amount of motion stabilization required to be set for the motion stabilization device based on the collected information, and
output the determined amount of motion stabilization.

2. The accessibility diagnosis system according to claim 1, wherein the input of a specific drawing motion includes at least one of drawing a straight line and drawing a circular pattern.

3. The accessibility diagnosis system according to claim 1, wherein the input of a specific movement of the mobile user device includes at least one of moving the mobile user device close to the user's face and rotating the mobile user device in a predetermined direction.

4. The accessibility diagnosis system according to claim 1, wherein the mobile user device is configured to provide a questionnaire to the user and the amount of motion stabilization required to be set for the motion stabilization device is further based on the user's responses to the questionnaire.

5. The accessibility diagnosis system according to claim 1, wherein the processing circuitry of the server device is configured to combine the collected information from the mobile user device and determine if motion stabilization is active or static based on comparing a value associated with the collected information to a threshold.

6. The accessibility diagnosis system according to claim 1, wherein the processing circuitry of the server device is configured to determine if motion stabilization is active or static for at least one particular cosmetic application based on comparing a value associated with the collected information to a threshold.

7. The accessibility diagnosis system according to claim 1, wherein the processing circuitry server device determines the amount of motion stabilization by inputting the collected information into a neural network that is trained to output a motion stabilization profile based on pre-existing combinations collected information the motion stabilization profiles.
